(19) United States
(12) Patent Application Publication
(10) Pub. No.: US 2010/0048687 A1
(43) Pub. Date: Feb. 25, 2010

(54) USE OF PHOSPHATASE INHIBITORS FOR THE TREATMENT OF NEURODEGENERATIVE DISEASES

(76) Inventors: Roger Nitsch, Zumikon (CH); Marlen Knobloch, Zurich (CH); Mélissa Farinelli, Zurich (CH); Isabelle Mansuy, Zurich (CH)

Correspondence Address: LATIMER & MAYBERRY IP LAW, LLP, 13873 PARK CENTER ROAD, SUITE 106, HERNDON, VA, 20171, US

(21) Appl. No.: 12/524,011

(22) PCT Filed: Jul. 22, 2008
(86) PCT No.: PCT/EP08/00469

§ 371 Date: Oct. 26, 2009

(30) Foreign Application Priority Data
Jan. 22, 2007 (EP) 07001345.3

(51) Int. Cl. A61K 31/352; C12Q 1/68; A61P 25/00

(52) U.S. Cl. 514/451; 435/6

(57) ABSTRACT

Provided are novel a target and drugs in the treatment of neurological disorders related to amyloid beta pathology/amyloidosis. More specifically, the use of phosphatase inhibitors for the treatment of brain impairments mediated by Aβ-oligomers is described.

### FIELD OF THE INVENTION

The present invention relates to the technical field of neurological disorders and methods for the treatment of the same. More specifically, the present invention pertains to the treatment of disorders mediated by the amyloidogenic pathway of the amyloid protein precursor (APP) and by amyloid beta (Aβ) oligomer aggregation in particular.

### BACKGROUND OF THE INVENTION

Oligomeric aggregates of amyloid beta (Aβ) peptide can disrupt synaptic plasticity and accumulate in brains of patients with Alzheimer's disease (AD) (Wirths et al., J. Neurochem. 91 (2004), 513-520; Oddo et al., Neuron 39 (2003), 409-421; Walsh et al., Nature 416 (2002), 535-539; Lesne et al., Nature 440 (2006), 352-357). Immunotherapy directed against Aβ has shown promising initial benefits in AD patients and rodent models (Hock et al., Neuron 38 (2003), 547-554; Klyubin et al., Nat. Med. 11 (2005), 556-561), but besides possible roles of Aβ in the disruption of calcium homeostasis (Mattson and Chan, Cell Calcium 34 (2003), 385-397) and specific interactions with receptors such as NMDA-(Snyder et al., Nat. Neurosci. 8 (2005), 1051-1058) and a-7 nicotinic acetylcholine receptors (Oddo and LaFerla, J. Physiol. Paris 99 (2006), 172-179), little is known about intracellular signaling pathways that couple Aβ toxicity to synaptic functions.

### SUMMARY OF THE INVENTION

The present invention relates generally to the use of agents capable of modulating protein phosphorylation in the treatment, amelioration and prevention, respectively, of neurological disorders, in particular disorders associated with Alzheimer's disease or related diseases with amyloid beta (Aβ) pathology and amyloidosis. In particular, the present invention makes use of the surprising finding that Aβ-oligomer mediated impairment of the brain can be reversed by modulating phosphorylation events by inhibiting protein phosphatase 1 (PP1); see appended Examples 2 and 3.

Besides the impact of the findings obtained in accordance with the present invention on approaching Alzheimer's disease and disorders related thereto, the present invention also provides novel diagnostic markers for the diagnosis of such disorders, i.e. phosphatase and kinase gene products, respectively, in particular PP1. In this context, the present invention also pertains to diagnostic compositions and kits for use in corresponding diagnostic methods employing genes and gene products involved in phosphorylation events as a diagnostic marker for Aβ pathology/amyloidosis.

In a further aspect, the present invention relates to a non-human transgenic animal that expresses human APP carrying at least two mutations, one or both of which are selected from familiar Swedish and Arctic mutations, for use in screening and profiling, respectively, a drug for the treatment of a neurological disorder such as Alzheimer's disease or a disorder associated therewith.

Other embodiments of the invention will be apparent from the description that follows.

### BRIEF DESCRIPTION OF THE DRAWINGS

FIG. 1: LTP impairment in arcAβ mice is partially rescued by passive immunization. FIG. 1a-1b) CA1 hippocampal LTP is severely impaired in slices from 3.5 and 7.5 months-old arcAβ mice (n=5 tg, 5 wt mice) but basal transmission is normal (inset). FIG. 1c) LTP and basal transmission (inset) are normal in slices from 1 month-old arcAβ mice (n=5 tg, 5 wt mice). FIG. 1d) Passive immunization with an anti-Aβ antibody partially rescues the LTP deficit in slices from 3.5 months-old arcAβ mice (85%±4.5% level of potentiation of wt slices) whereas immunization with a control antibody has no effect (n=3 tg mice immunized with anti-Aβ antibody, n=3 tg mice immunized with control antibody). FIG. 1e) Real time RT-PCR: normal mRNA expression of NMDA and AMPA receptors, CaMKII and synaptophysin but reduced zif268 (52%, range 45-61%) and arg3.1 expression (67%, range 58-78%, n=5 tg and 5 wt mice) in the hippocampus of 6 months-old arcAβ mice. Error bars=s.e.m.

FIG. 2: In vivo and in vitro inhibition of PP1 reverses Aβ-oligomer-mediated LTP impairment. FIG. 2a-2b) PP1 is inhibited by bath-application of 1 nM tautomycin in slices of 3 months old arcAβ mice and wt littermates. Tautomycin fully rescues the LTP deficit in slices from tg mice (n=3 tg mice) but has no effect on wt slices (n=3 wt mice). FIG. 2c) Phosphatase activity assays showing that 1 nM tautomycin specifically inhibits the activity of recombinant PP1 but not calcineurin. FIG. 2d) Aβ-oligomers are produced from synthetic Aβ 1-42 according to Klein (Klein, Neurochem. Int. 41 (2002), 345-352). Electron microscopy of Aβ-oligomers preparation shows globular Aβ-assemblies of 7-12 nm after 24 h incubation at 4° C. Scale bar=100 nm. FIG. 2e-2f) Aβ-oligomers are bath-applied for at least 1 h to slices from I-1 * control mice and I-1* transgenic mice expressing an endogenous inhibitor of PP1. Aβ-oligomers impair LTP in I-1* control mice (n=5) but not in mutant mice (n=5).

FIG. 3: Reduced PP1 activity confers resistance to Aβ-oligomer mediated LTP impairment. FIG. 3a) Representative traces of fEPSPs before (upper line) and after LTP induction (lower line) in slices from 1 months, 3.5 months and 7.5 months-old arcAβ mice (tg) and littermate controls (wt). FIG. 3b) Verification of Aβ-oligomer mediated toxicity in wildtype mice. Aβ-oligomers were bath-applied to wildtype slices for at least 1 h. Aβ-oligomers impair LTP (n=2, 3 slices with Aβ-oligomers, 2 control slices).

### DEFINITIONS

Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2.

“Level”, as the term is used herein, generally refers to a gage of, or a measure of the amount of, or a concentration of a transcription product, for instance an mRNA, or a translation product.

“Activity”, as the term is used herein, generally refers to a measure for the ability of a transcription product or a translation product to produce a biological effect or a measure for a level of biologically active molecules. The terms “level” and/or “activity” as used herein further refer to gene expression levels, gene activity, or enzyme activity.

“Modulator”, as the term is used herein, generally refers to a molecule capable of changing or altering the level and/or the activity of a gene, or a transcription product of a gene, or a translation product of a gene. Preferably, a “modulator” is capable of changing or altering the biological activity of a transcription product or a translation product of a gene. Said modulation, for instance, may be an increase or a decrease in enzyme activity, a change in binding characteristics, or any other change or alteration in the biological, functional, or immunological properties of said translation product of a gene.

“Oligonucleotide primer” or “primer”, as the terms are used herein, generally refer to short nucleic acid sequences which can anneal to a given target polynucleotide by hybridization of the complementary base pairs and can be extended by a polymerase. They may be chosen to be specific to a particular sequence or they may be randomly selected, e.g. they will prime all-possible sequences in a mix. The length of primers used herein may vary from 10 nucleotides to 80 nucleotides.

“Probes”, as the term is used herein, generally refers to short nucleic acid sequences of the nucleic acid sequences of phosphatases and kinases referred to, described and/or disclosed herein or sequences complementary therewith. They may comprise full length sequences, or fragments, derivatives, isoforms, or variants of a given sequence. The identification of hybridization complexes between a “probe” and an assayed sample allows the detection of the presence of other similar sequences within that sample.

“Agent”, “reagent”, or “compound”, as the terms are used herein, generally refer to any substance, chemical, composition, or extract that have a positive or negative biological effect on a cell, tissue, body fluid, or within the context of any biological system, or any assay system examined. They can be agonists, antagonists, partial agonists or inverse agonists of a target. Such agents, reagents, or compounds may be nucleic acids, natural or synthetic peptides or protein complexes, or fusion proteins. They may also be antibodies, organic or inorganic molecules or compositions, small molecules, drugs and any combinations of any of said agents above. They may be used for testing, for diagnostic or for therapeutic purposes.

If not stated otherwise, the terms “compound”, “substance” and “(chemical) composition” are used interchangeably herein and include but are not limited to therapeutic agents (or potential therapeutic agents), food additives and nutraceuticals. They can also be animal therapeutics or potential animal therapeutics.

“Small organic molecule”, as the term is used herein, refers to an organic compound [or organic compound complexed with an inorganic compound (e.g., metal)] that has a molecular weight of less than 3 kilodaltons, preferably less than 1.5 kilodaltons. Furthermore, the term “synthetic organic molecule” may be used interchangeably with the term “small organic molecule” except that the synthetic organic molecule is made by man and not to be found in nature unless stated otherwise.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

Furthermore, the term “subject” as employed herein relates to animals in need of therapy, e.g. amelioration, treatment and/or prevention of Aβ mediated disorder. Most preferably, said subject is a human.

General Techniques

For further elaboration of general techniques useful in the practice of this invention, the practitioner can refer to standard textbooks and reviews in cell biology and tissue culture; see also the references cited in the examples. General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Non-viral Vectors for Gene Therapy (Wagner et al. eds., Academic Press 1999); Viral Vectors (Kaplitt & Loewy eds., Academic Press 1995); Immunology Methods Manual (Lefkovits ed., Academic Press 1997); and Cell and Tissue Culture: Laboratory Procedures in Biotechnology (Doyle & Griffiths, John Wiley & Sons 1998). Reagents, cloning vectors and kits for genetic manipulation referred to in this disclosure are available from commercial vendors such as BioRad, Stratagene, Invitrogen, Sigma-Aldrich, and ClonTech.

### DETAILED DESCRIPTION OF THE INVENTION

The present invention generally relates to means and methods for the treatment of neurological disorders and Alzheimer's disease in particular. More specifically, as disclosed in Examples 2 and 3, it could surprisingly be shown that modulation of phosphorylation and dephosphorylation activity, respectively, provides a therapeutic approach for the treatment, amelioration and prevention of impairment of brain function. The present invention is based on the observation that Aβ-oligomer mediated impairment of hippocampal long term potentiation (LTP) is rescued by protein phosphatase 1 (PP 1) inhibition.

Without intending to be bound by theory it is, thanks to the experiments performed in accordance with the present invention, believed that Aβ-oligomers are toxic APP by-products that impair memory and hippocampal long-term potentiation (LTP) in vivo and in vitro. In accordance with the present invention it could surprisingly be shown that Aβ-induced LTP impairment involves protein phosphatase 1 (PP1)-dependent mechanisms. It can be partially rescued by passive immunization against Aβ and fully by PP1 inhibition; see Example 2. Furthermore, it was demonstrated that endogenous PP1 inhibition in vivo confers resistance to Aβ-oligomer toxicity, revealing PP1 and thus phosphorylation events as a key player in AD pathology as well as probably other neurological disorders; see Example 3. The findings of the present invention have been confirmed and published by the inventors in Knobloch et al., J. Neuroscience 27 (2007), 7648-7653, the disclosure content of which is incorporated herein by reference.

Previously, assaying the presence of PP-1 as well as kinases and other phosphatases have been suggested to be monitored in context with Alzheimer tau protein phosphorylation which is thought to be involved in paired helical formation from tau protein in Alzheimer disease; see European patent application EP 0 911 390 A2. Furthermore, PP1 inhibition was shown to reverse cognitive deficits in aged mice (Genoux et al., Nature 418 (2002), 970 975). However, PP1 as a marker and target, respectively, for Aβ-oligomer mediated toxicity has not been considered.

In contrast, more recently PP-1 inhibitors have been suggested for use to prevent missplicing events in various pathological situations, including degenerative diseases and cancers; see European patent application EP 1 736 154 A1.

Hence, the beneficial effect of PP-1 inhibitors on Aβ oligomer-mediated toxicity has not been envisaged. Thus, the observations made in accordance with the present invention significantly extend the previous findings and contribute novel diagnostic and therapeutic means to approach Aβ oligomer-mediated toxicity.

Accordingly, the present invention relates to the use of an agent capable of modulating protein phosphorylation, i.e. by inhibiting protein phosphatase 1 (PP1) for the preparation of a pharmaceutical composition for the treatment, amelioration or prevention of a neurological disorder, in amyloid β (Aβ) pathology/amyloidosis.

The pharmaceutical compositions of the present invention can be formulated according to methods well known in the art; see for example Remington: The Science and Practice of Pharmacy (2000) by the University of Sciences in Philadelphia, ISBN 0-683-306472. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intra-muscular, topical or intradermal administration. Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as dopamine or psychopharmacologic drugs, depending on the intended use of the pharmaceutical composition. Furthermore, the pharmaceutical composition may also be formulated as a vaccine, for example, if the pharmaceutical composition of the invention comprises an anti-Aβ antibody for passive immunization.

In addition, co-administration or sequential administration of other agents may be desirable. A therapeutically effective dose or amount refers to that amount of the active ingredient sufficient to ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

Preferably, the therapeutic agent in the composition is present in an amount sufficient to rescue Aβ-oligomer mediated impairment of hippocampal LTP.

The pharmaceutical compositions in accordance with the present invention can be used for the treatment of neurological disorders including but not limited to Alzheimer's disease, aphasia, Bell's Palsy, Creutzfeldt-Jakob disease, epilepsy, encephalitis, Huntington's disease, neuromuscular disorders, neuro-oncology, neuro-immunology, neuro-otology pain, pediatric neurology, phobia sleep disorders, Tourette Syndrome, Parkinson's disease, other movement disorders and disease of the central nervous system (CNS) in general.

As demonstrated in the examples, the present invention particularly provides a method of treating and preventing, respectively, Alzheimer's disease and disorders associated therewith comprising administering to a subject in need thereof or supposed to become in need a therapeutically effective amount of a pharmaceutical composition comprising the agent capable of modulating protein phosphorylation. Said agents are further characterized below.

As demonstrated in the examples, inhibiting phosphatase activity, in particular of protein phosphatase 1 (PP1), resulted in the reversion and prevention, respectively, of impairment of brain function of mice which were induced to develop a neurological disorder.

Protein phosphatase 1 (PP1) is a major eukaryotic protein serine/threonine phosphatase that regulates an enormous variety of cellular functions through the interaction of its catalytic subunit (PP1c) with over fifty different established or putative regulatory subunits. The nucleotide and amino acid sequences of the human serine/threonine-protein phosphatases are known in the art and can be obtained via public databases, for example the internet pages hosted by the National Center for Biotechnology Information (NCBI), including the NIH genetic sequence database GeneBank, which also cites the corresponding references available by PubMed Central. For example, the human nucleotide and amino acid sequences of PP1-α catalytic subunits are available under primary Accession number P62136; of the PP1-β catalytic subunit under primary Accession number P62140; and of the PP1-γ catalytic subunit under primary Accession number P36873. The corresponding nucleotide and amino acid sequences of mouse PP1 catalytic subunits are available under Accession numbers P62137, P62141 and P63087. Furthermore, PP1-γ1 and PP1-γ2 catalytic subunits are known which however represent alternatively spliced isoforms generated from a single gene. Most of these target PP1c to specific subcellular locations and interact with a small hydrophobic groove on the surface of PP1c through a short conserved binding motif—the RVxF (SEQ ID NO: 17) motif—which is often preceded by further basic residues. Weaker interactions may subsequently enhance binding and modulate PP1 activity/specificity in a variety of ways. Several putative targeting subunits do not possess an RVxF motif but nevertheless interact with the same region of PP1c. In addition, several “modulator” proteins bind to PP1c but do not possess a domain targeting them to a specific location. Most are potent inhibitors of PP1c and possess at least two sites for interaction with PP1c, one of which is identical or similar to the RVxF motif. Regulation of PP1c in response to extracellular and intracellular signals occurs mostly through changes in the levels, conformation or phosphorylation status of targeting subunits. The mode of action of PP1c complexes facilitates the development of drugs that target particular PP1c complexes and thereby modulate the phosphorylation state of a very limited subset of proteins. For review see, e.g., Cohen, J. Cell Sci. 115 (2002), 241-256. Thus, an agent capable of modulating protein phosphorylation, especially phosphatase modulators can be based on and/or directed to the interaction of the enzyme, e.g., phosphatase 1 with any one of its regulatory subunits, most preferably those that bind and are preferably specific for the mentioned binding motif RVxF. Such modulators may interfere with complex formation of protein phosphatase complexes and/or targeting the protein phosphatase and complex, respectively, to its native subcellular location. Such modulators and in particular inhibitors are advantageous, since they are more specific than an agent which affects the catalytic activity of the enzyme only. In this context, it is also to be understood that agents useful according to the present invention rather than being directed to the protein phosphatase can be specific for a binding partner of the enzyme such as one of the regulatory subunits which are for example necessary for the enzyme to exert its enzymatic activity and/or correct subcellular location.

Hence, phosphatase inhibitors, in particular PP1 inhibitors are preferably used in the pharmaceutical compositions of the present invention. However, other phosphorylation modulators may be used as well. In this context, the modulators of phosphorylation, in particular phosphatase inhibitors, are well known in the art and include, for example, okadaic acid, microcystins, nodularin, cantharidin, calyculin A, tautomycin, and fostriecin. Furthermore, the development of chimeric antisense oligonucleotides that support RNAase H mediated degradation of the targeted mRNA has resulted in compounds capable of specifically suppressing the expression of PP5 (ISIS 15534) and PP1gamma 1 (ISIS 14435) in human cells. Such compounds have already proven useful for the validation of drug targets, and if difficulties associated with systemic delivery of antisense oligonucleotides can be overcome, antisense is poised to have a major impact on the clinical management of many human diseases. A corresponding antisense approach for inhibiting protein phosphatase expression in methods of treating cancer has been described in international application WO99/27134. In addition, methods for screening for modulators of kinase or phosphatase activity are well known in the art and described, for example, in international application WO01/25477. Hence, approaches and agents hitherto used for modulating phosphorylation activity, in particular for inhibiting phosphatase activity, may be used and employed in the pharmaceutical compositions and therapeutic treatments of the present invention.

As described in the examples, the therapeutic agent, here PP1 inhibitor, can be applied exogenously to (Example 2) or expressed in a target cell or tissue (Example 3).

In one preferred embodiment, the agent capable of modulating phosphorylation in accordance with the present invention is a drug which can be formulated into pharmaceutical compositions in accordance methods well known in the art, see also supra. Such agents capable of modulating phosphorylation are well known in the art; see, for example, the phosphatase inhibitors mentioned above, and further compounds with phosphatase inhibitory activity are still being identified. For example, tautomycetin, a natural phosphatase inhibitor, has been isolated and described by Mitsuhashi et al., Biochem. Biophys. Res. Commun. 287 (2001), 328-331. In accordance with the present invention, tautomycin, which has been proven to be effective in the animal model, is preferred to be used in the pharmaceutical compositions of the present invention.

PP1-inhibitory proteins, which can be expressed in a given target cell or tissue, in particular in the brain, are also well known in the art. For example, GBPI, a gastrointestinal- and brain-specific PP1-inhibitory protein, has been described by Liu et al., Biochem. J. 377 (2004), 171-181 and KEPI, a protein kinase C (PKC)-potentiated inhibitor protein for PP1 has been described by Liu et al. in J. Biol. Chem. 277 (2002), 13312-13320. Further, the cDNA sequence encoding inhibitor-2 of PP1 has been described in international application WO02/056837. Most preferably, however, PP1 inhibitor (I-1*), which has been demonstrated to prevent the onset of a neurological disorder in mice which were exposed to Aβ-oligomers, is preferably used for gene-therapeutic approaches in accordance with the present invention. Meanwhile gene technology-based therapies in the brain have been established for disorders including Alzheimer's disease, Parkinson's disease and brain neoplasms; see for review Wirth and Yla-Herttuala, Adv. Tech. Stand. Neurosurg. 31 (2006), 3-32. For example, lentivirus-mediated gene transfer to the central nervous system in order to provide effective long-term treatment of neurological disorders such as Parkinson's disease, Alzheimer's disease, Huntington's disease, motor neuron diseases, lysosoma storage diseases and spinal injury have been reported; see, for example, the summary in Wong et al., Hum. Gene Ther. 17 (2006), 1-9. Likewise, suppression of expression of a kinase or phosphatase gene product such as PP1 may be achieved via antisense or RNAi technology. For example, herpes simplex virus RNAi and neprilysin gene transfer vectors for down-regulation of APP have been reported by Hong et al., Gene Ther. 13 (2006), 1068-1079. In addition, stem cell strategies may be applied in accordance with the gene-therapeutic approaches of the present invention. For example, human neural stem cells (HNSCs) can be transplanted into the brain, which differentiate into neural cells and significantly improve cognitive functions; see, for example, Sugaya et al., Panminerva Med. 48 (2006), 87-96. Such stem cells may be applied in combination with either the gene-therapeutic approach described above or a therapeutic agent based on a small organic compound such as tautomycin. Of course, in one embodiment such stem cell may be genetically engineered with a therapeutic gene, in particular a gene product which is capable of modulating phosphorylation, preferably the activity of PP1, in order to render the neural cells developing from said stem cells resistant to the deleterious effects of Aβ oligomers Thus, the findings of the present invention may find their way in various therapeutic approaches, which so far are based on the use of different therapeutic genes.

The person skilled in the art will therefore acknowledge that there are various ways in order to put the present invention into practice and that the means therefore are substantially unlimited. Thus, the agent capable of modulating phosphorylation, in particular phosphatase activity, can be of any kind and includes, for example, an agent selected from the group consisting of an antisense nucleic acid, siRNA, a ribozyme, an antibody, a peptide, a peptide mimetic or a small and synthetic organic molecule, respectively.

In a further aspect, the present invention relates to a method of diagnosis of a disorder associated with Alzheimer's disease, more particularly a disorder associated with amyloid β (Aβ) pathology/amyloidosis, e.g. impairment of hippocampal long-term potentiation (LTP), said method comprising:
(a) assaying a sample from a subject for phosphatase or kinase gene product or activity; and
(b) determining the level of phosphatase or kinase gene product or activity, wherein an altered level compared to a control indicates the presence of the disorder.

In particular, an increased level of phosphatase expression and activity, respectively, compared to a healthiness control may be indicative for the presence of the disorder. On the other hand, for the kinase gene product a decreased level of expression and activity, respectively, may be decisive for the presence of the disorder. Alternatively, or in addition, a sample from a subject known to suffer from a neurological disorder such as Alzheimer's disease and having an altered level of phosphatase and/or kinase gene product or activity, is used as a positive control. In this embodiment, the test subject may be tested positively, if the level of expression or activity of a given phosphatase or kinase gene product substantially matches that of the positive control. In this context, it is of course advantageous to determine the level of expression or activity of more than one phosphatase or kinase gene. For this embodiment, microarray and chip technology is particularly suited. Preferably, at least the level of expression or activity of PP1 is determined.

In one embodiment, the phosphatase or kinase gene product is determined by a nucleic acid, wherein the nucleic acid is preferably labeled or otherwise modified. Furthermore, microarray and chip technology may be used for determining the level of phosphatase or kinase gene expression.

The use of microarrays in analyzing gene expression is reviewed generally by Fritz et al., Science 288 (2000), 316; Microarray Biochip Technology, www.Gene-Chips.com. An exemplary method is conducted using a Genetic Microsystems array generator, and an Axon GenePix Scanner. Microarrays are prepared by first amplifying cDNA fragments encoding marker sequences to be analyzed, and spotted directly onto glass slides to compare mRNA preparations from two cells of interest, one preparation is converted into Cy3-labeled cDNA, while the other is converted into Cy5-labeled cDNA. The two cDNA preparations are hybridized simultaneously to the microarray slide, and then washed to eliminate non-specific binding. The slide is then scanned at wavelengths appropriate for each of the labels, the resulting fluorescence is quantified, and the results are formatted to give an indication of the relative abundance of mRNA for each marker on the array.

Alternatively, the phosphatase or kinase gene product is determined by an antibody selected from the group consisting of a polyclonal antibody, a monoclonal antibody, a human antibody, humanized antibody, a chimeric antibody, and a synthetic antibody. Preferably, the antibody is detectably labeled or otherwise modified and/or to be detected by a secondary antibody.

Generally, methods for detecting protein kinase and protein phosphatase expression or activity are well known to the person skilled in the art and can be found in such standard textbooks; see also supra and the appended examples. Reagents, detection means and kits for diagnostic purposes are available from commercial vendors such as Pharmacia Diagnostics, Amersham, BioRad, Stratagene, Invitrogen, and Sigma-Aldrich as well as from the sources given any one of the references cited herein, in particular patent literature. For example, international application WO2006/014645 describes generic probes that bind to phosphorylated amino acid residues as well as methods employing the probes for screening for kinase inhibitory activity, kinase activity, and phosphatase activity. Methods for distinguishing serine/threonine kinase phosphorylation from tyrosine kinase phosphorylation are also provided. In addition, international application WO2006/083016 and European patent application EP 1 199 370 describe means and methods for determining the activity of protein kinase and protein phosphatase, respectively, making use of peptide substrate and immunoassay techniques employing inter alia antibodies having a specificity to the substrate peptide or protein that is phosphorylated. The disclosure content of any one of those applications is incorporated herein by reference in their entirety, in particular with respect to the nucleic acid and antibody probes as well as reagents for detecting kinase and phosphatase activity for use in the diagnostic methods and kits therefore of the present invention.

Hence, the present invention also relates to a kit for use in any one of the above-described diagnostic methods, said kit comprising appropriate reagent means such as those described in the Protein Tyr Phosphatase (PTP) Assay System of New England Biolabs Inc. or the PP1/PP2A Toolbox of Upstate Inc., cell signaling solutions. In addition, or alternatively, the kit comprises an appropriate antibody and/or nucleic acid molecule, as mentioned before, which are specific for the phosphatase and encoding mRNA/cDNA, respectively. Suitable reagents include, but are not limited to, PP1 enzyme, PP2A enzyme, PHI-1 protein, okadaic acid, calyculin A, protein phosphatase dilution buffer, BSA, etc.

In a further aspect, the present invention relates to a non-human transgenic animal, preferably mouse that expresses human APP carrying both familial Swedish and Arctic mutations such as described in Knobloch et al., Neurobiol. Aging July 28 (2006), S1558-1497 (epublished ahead of print), for use in screening or profiling a drug for the treatment of a neurological disorder, preferably Alzheimer's disease or a disorder associated therewith. As demonstrated in the examples, such non-human transgenic animal is particularly useful in investigating drug- as well as gene-therapeutic approaches for the treatment of Alzheimer's disease, in particular with respect to Aβ-oligomer mediated LTP.

Furthermore, developing a drug based on the agent capable of modulating protein phosphorylation has been proven useful in accordance with the present invention, including obtaining marketing authorization and actually putting the authorized drug on the market can be achieved by a different company. Thus, in a further aspect the present invention relates to a method of conducting a drug development business comprising licensing, to a third party, the rights for further drug development and/or sales for therapeutic agents identified or profiled in accordance with the present invention, or analogs thereof.

For suitable lead compounds that have been provided, further profiling of the agent, or analogs thereof, can be carried out for assessing efficacy and toxicity in animals, depending on the modalities of the agreement with the respective third party. Further development of those compounds for use in humans or for veterinary uses will then be conducted by the third party. The subject business method will usually involve either the sale or licensing of the rights to develop said compound but may also be conducted as a service, offered to drug developing companies for a fee.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. Several documents are cited throughout the text of this specification. Full bibliographic citations may be found at the end of the specification immediately preceding the claims. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

A more complete understanding can be obtained by reference to the following specific examples which are provided herein for purposes of illustration only and are not intended to limit the scope of the invention.

### Examples

The examples which follow further illustrate the invention, but should not be construed to limit the scope of the invention in any way. Detailed descriptions of conventional methods, such as those employed herein can be found in the cited literature; see also “The Merck Manual of Diagnosis and Therapy” Seventeenth Ed. ed by Beers and Berkow (Merck & Co., Inc. 2003).

The practice of the present invention will employ, unless otherwise indicated, conventional techniques of cell biology, cell culture, molecular biology, transgenic biology, microbiology, recombinant DNA, and immunology, which are within the skill of the art.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Reagents, cloning vectors, and kits for genetic manipulation referred to in this disclosure are available from commercial vendors such as BioRad, Stratagene, Invitrogen, and Clontech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11 (2001), 98-107.

Supplementary Methods

Animals

ArcAβ mice and I-1* mutant mice were obtained by breeding as previously described (Knobloch et al., Neurobiol. Aging, July 28 (2006), S1558-1497; Genoux et al., Nature 418 (2002), 970-975; Michalon et al., Genesis 43 (2005), 205-212). ArcAβ mice express human APP695 carrying both the Swedish (K670N; M671L) and the Arctic (E693G) mutations in a single construct under the control of the prion protein promoter. I-1* mutant mice express an rtTA2 transgene under the control of the CaMKIIalpha promoter and an I1* transgene under the control of a tetO promoter. Mice were kept under standard housing conditions on a reversed 12 h: 12 h light/dark cycle with food and water ad libitum. 7-9 days before the experiments, I-1* mutant mice and control littermates (carrying only the tetO-I-1* transgene) were fed doxycycline (Westward Pharmaceuticals) at 6 mg/g food. All animal experiments were performed in accordance with guidelines of the Swiss veterinary cantonal office (licenses Nr 108/03 and Nr 123/04).

Electrophysiological Recordings

Mice were anaesthetized with isofluran then decapitated. Heads were immediately immersed in ice-cold freshly prepared artificial cerebrospinal fluid (aCSF) for at least 2 min before brain extraction. Acute slices (400 μm thick) were prepared with a vibratome (Leica VT 1000S) in ice-cold gazed aCSF. Sections were incubated in aCSF at 34° C. for 20 min then kept at room temperature for at least 1 hour before recording. Recording was performed in an interface chamber continuously flowed with aCSF at 1.1 ml/min. A monopolar electrode was placed in the Schaffer collaterals and stimulation was applied at 0.033 Hz with stimulus intensity ranging from 20-80 μA depending on the size of the evoked field excitatory postsynaptic potentials (fEPSPs). fEPSPs were recorded in the stratum radiatum with a borosilicate micropipette filled with aCSF. The signal was amplified with an AXOPATCH 200B amplifier (Axon Instruments), digitized by a digidata 1200 interface (AxonInstruments) and sampled at 10 kHz with Clampex 8.2 (AxonInstruments).

aCSF composition (mM): NaCl, 119, D-glucose, 11, MgCl₂.6H₂O, 1.3, NaH₂PO₄, 1.3, KCl, 2.5, CaCl₂, 2.5, NaHCO₃, 26, gazed with O₂/CO₂ (95/5%) at least 20 min before use throughout the experiment.

LTP induction: LTP was induced after minimum 20-min of stable baseline using 3 trains of 100 Hz tetanus delivered at stimulus intensity, separated by 20 seconds.

Data analysis: For each experiment, the slope of individual fEPSPs was measured in the linear 1-1.5 ms-portion by linear fitting using Clampfit (Axon Instruments).

Reverse Transcription and Quantitative Real-Time PCR

Total RNA was isolated from frozen hippocampi of 6 month-old arcAβ mice and wt littermates (n=5 for each) with TRIzol (Invitrogen) and cleaned up with RNeasy Mini Kit (Quiagen). First-strand cDNA was synthesized using the SuperScript First-Strand Synthesis System for RT-PCR (Invitrogen) according to the manufacturer's protocol. Real-time-PCR was performed on TaqMan (ABI PRISM™ 7700 SDS) using SYBR Green (Applied Biosystems). Primer Sequences: forward (for) and reverse (rev)

| NR2B | | |
|---|---|---|
| for.: | AAGACAAGGGCCGATTCATG | (SEQ ID NO: 1) |
| rev.: | GCAAAGGAGCTCTCACCAGC | (SEQ ID NO: 2) |
| CaMKII | | |
| for.: | AGTCAGAGGAGACCCGCGT | (SEQ ID NO: 3) |
| rev.: | TGTGGAAGTGGACGATCTGC | (SEQ ID NO: 4) |
| GluR1 | | |
| for.: | CAATGTGGCAGGCGTGTTC | (SEQ ID NO: 5) |
| rev.: | TCGATTAAGGCAACCAGCATG | (SEQ ID NO: 6) |
| Syn.physin | | |
| for.: | AAGGTGCTGCAGTGGGTCTTT | (SEQ ID NO: 7) |
| rev.: | CGAAGCTCTCCGGTGTAGCT | (SEQ ID NO: 8) |
| Zif26 | | |
| for.: | CGAGAAGCCTTTTGCCTGTG | (SEQ ID NO: 9) |
| rev.: | TGGTATGCCTCTTGCGTTCA | (SEQ ID NO: 10) |
| Arc | | |
| for.: | TGGAGGGAGGTCTTCTACCGT | (SEQ ID NO: 11) |
| rev.: | TATTTGCCGCCCATGGACT | (SEQ ID NO: 12) |
| β-actin | | |
| for.: | TACTCTGTGTGGATCGGTGGC | (SEQ ID NO: 13) |
| rev.: | TGCTGATCCACATCTGCTGG | (SEQ ID NO: 14) |
| GAPDH | | |
| for.: | GGCATCTTGGGCTACACTGAG | (SEQ ID NO: 15) |
| rev.: | CGAAGGTGGAAGAGTGGGAG | (SEQ ID NO: 16) |

Data analysis was performed according to the deltadelta Ct method, normalized to β-actin and GAPDH. Statistical analysis was performed on the deltaCt values using Student's t-test.

Passive Immunization

3 month-old arcAβ mice were immunized with a single intraperitoneal injection (10 mg/kg) of either purified 6E10 (Signet) or negative control antibody for mouse IgG₁ (Lab vision) 48 h before slice preparation.

Tautomycin Application

Slices from 3-month old arcAβ mice and wt littermates were bathed in normal aCSF or aCSF containing 1 nM tautomycin (Sigma) for at least 1 h before recording.

PP1 Activity Assays

Activity of recombinant PP1 (NEB) and calcineurin (Biomol) was measured with and without 1 nM tautomycin using the Biomol Green Assay-Kit (Biomol) according to manufacturer's instruction.

Aβ-oligomers Preparation and Application

Aβ-oligomers were prepared according to Klein (Klein, Neurochem. Int. 41 (2002), 345-352). Synthetic Aβ 1-42 (Bachem) was dissolved in Hexafluor2-propanol (HFIP), aliquoted and kept at −80° C. after evaporation of HFIP. Aβ-oligomers were prepared freshly by dissolving the above peptide film with DMSO and diluting it into cold F12 medium without phenol red to yield a 100 μM stock. This preparation was incubated at 4° C. for 24 h, centrifuged at 14'000 g for 10 min at 4° C. and supernatant was further used for electrophysiological experiments according to Wang et al. (Wang et al., Brain Res. 924 (2002), 133-140). Slices were bathed for at least 1 h in aCSF containing either Aβ-oligomers (1:200 diluted) or, as a control, phenolred-free F12 medium only (1:200 diluted). To prevent a wash out of Aβ-oligomers during recording, the aCSF used for perfusion contained a 1:400 dilution of Aβ-oligomers/phenolred-free F12 respectively. Each Aβ-oligomers preparation was used for one I-1* mutant mouse and a corresponding control in parallel, measured in a blinded fashion.

Electron Microscopy

The Aβ-oligomers preparation was controlled using electron microscopy. 5 μl of the above supernatant were adsorbed onto glow-discharged, 300-mesh carbon—coated Formvar grids for 2-3 min, negatively stained with 2% phosphorotungsten acid for 45 sec and viewed with a Philips CM12 scanning transmission electron microscope.

### Example 1

### Hippocampal Long Term Potentiation (LTP) is Mediated by Aβ

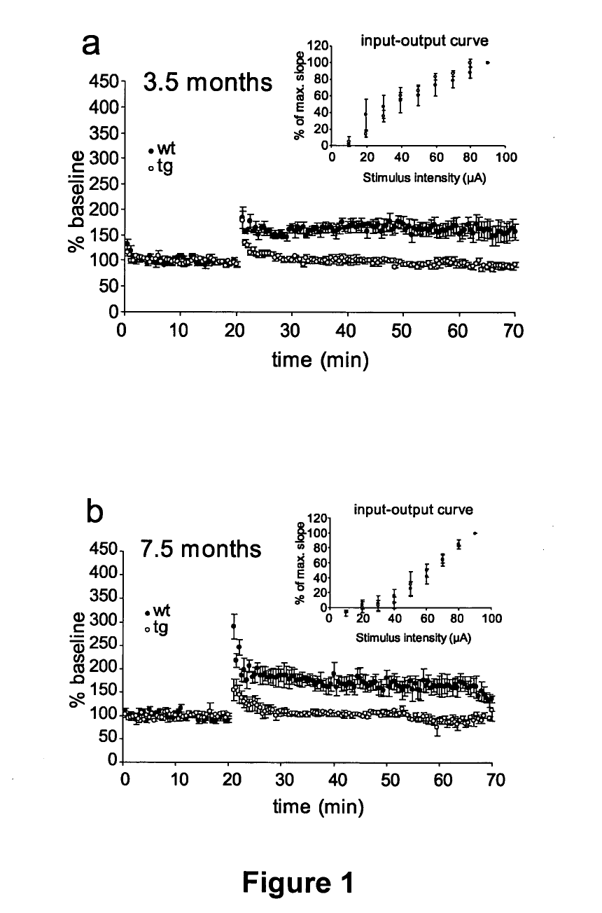

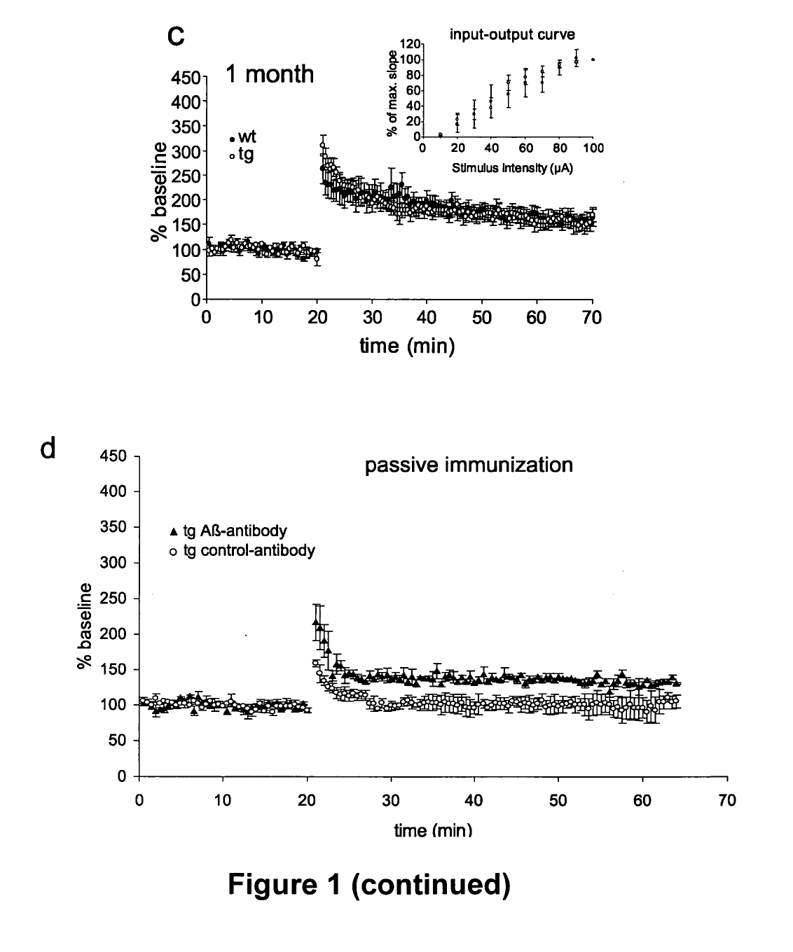

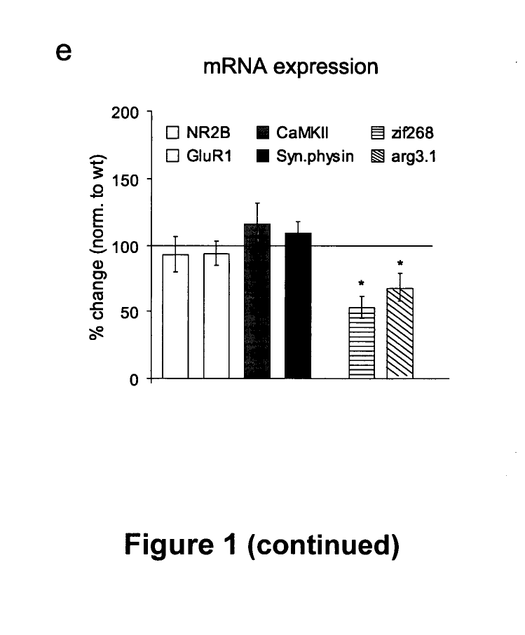

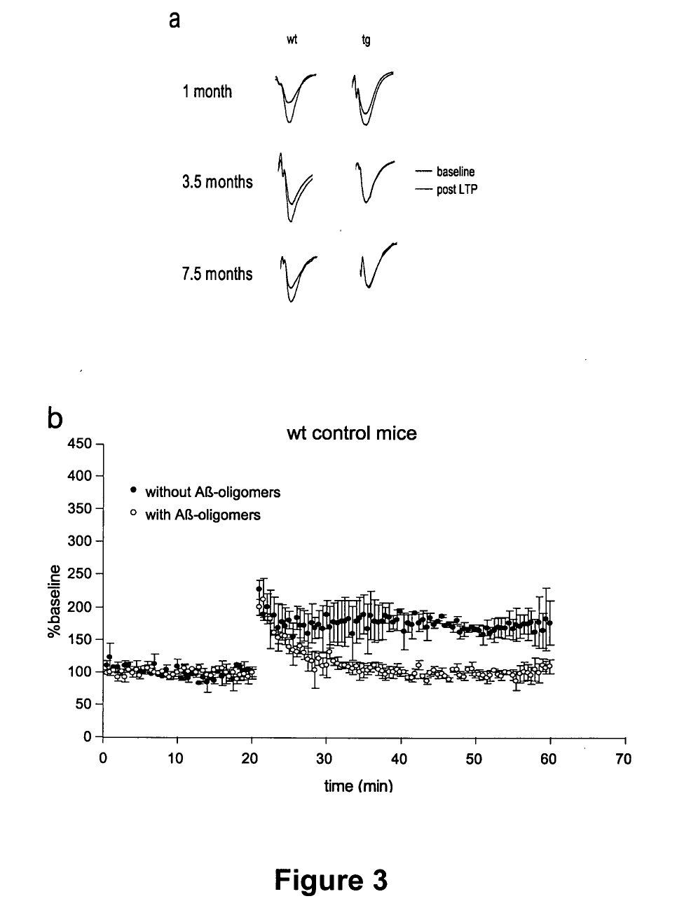

To determine the potential pathways underlying Aβ toxicity in vivo, hippocampal LTP in transgenic mice expressing human APP containing both Swedish and Arctic mutations (Knobloch et al., Neurobiol. Aging, July 28 (2006), S1558-1497) were examined. In these mice (arcAβ line), punctate intraneuronal Aβ deposits correlate with behavioral deficits before the onset of extracellular β-amyloid plaque deposition. Hippocampal LTP in area CA1 was measured in vitro by field excitatory postsynaptic potential (fEPSP) recordings (supplementary methods). LTP was severely impaired in slices from 3.5 and 7.5 months old arcAβ mice (FIGS. 1a and b, p<0.001, repeated measurement ANOVA), with fEPSPs returning to baseline within 10 min after LTP induction (representative traces, FIG. 3a). The LTP deficit was not caused by impaired synaptic transmission because basal transmission was normal in the transgenic mice (FIGS. 1a and b inlet). Further, it did not result from a developmental effect of transgene expression as both LTP (FIG. 1c) and basal synaptic transmission (FIG. 1c inset) were normal in slices from 1 month-old mice, with no detectable Aβ accumulation despite high mutant APP expression (Knobloch et al., Neurobiol. Aging, July 28 (2006), S1558-1497).

To demonstrate that the LTP deficit in arcAβ mice is mediated by Aβ, 10 mg/kg of 6E10, a monoclonal antibody directed against the Aβ sequence were administered to 3.5 month-old transgenic mice and measured LTP in hippocampal slices 48 h after passive antibody transfer. It was observed that the antibody partially rescued the LTP deficit (FIG. 1d, p<0.01, repeated measurement ANOVA; tg anti-Aβ antibody vs. non-treated tg), restoring a level of potentiation that was ˜85% that of control slices. To exclude a non-specific effect of the antibody, a murine antibody of the identical IgG class raised against an artificial synthetic hapten not present in mice was used. As expected, this antibody failed to improve LTP in arcAβ mice (FIG. 1d, p<0.05, repeated measurement ANOVA; tg anti-Aβ antibody vs. tg control antibody; p=0.6, tg control antibody vs. non-treated tg), showing that the LTP deficit arcAβ mice is Aβ-mediated.

### Example 2

### Protein Phosphatase 1 (PP1) is Involved in Aβ Mediated LTP

To characterize signaling mechanisms involved in Aβ-mediated LTP impairment, the expression level of candidate genes involved in early phases of synaptic signaling was examined using real time RT-PCR. Hippocampal expression of both NMDA and AMPA receptors, ionotropic receptors critical for glutamatergic neurotransmission, was not changed in arcAβ transgenic mice (FIG. 1e). Likewise, the expression of calcium/calmodulin-dependent kinase II (CaMKII), a major protein kinase in the post-synaptic density needed for the induction of LTP (Lisman et al., Nat. Rev. Neurosci. 3 (2002), 175-190), or synaptophysin, a synaptic vesicle protein involved in neurotransmitter release, was not changed in arcAβ transgenic mice (FIG. 1e). These results suggest no gross alteration in functional or structural properties that could explain the LTP impairment in arcAβ mice. However, the expression of two inducible transcription factors, arg3.1 and zif268, activated upon neuronal activity and required for LTP (Tischmeyer and Grimm, Cell Mol. Life Sci. 55 (1999), 564-574), was significantly reduced in arcAβ mice compared to wildtype littermates (FIG. 1e, p>0.05, t-test, % change and range: arg3.1=67% (58-78%), zif268=52% (45-61%)), suggesting that Aβ accumulation in these mice is associated with impaired synaptic signaling and related immediate early gene transcription.

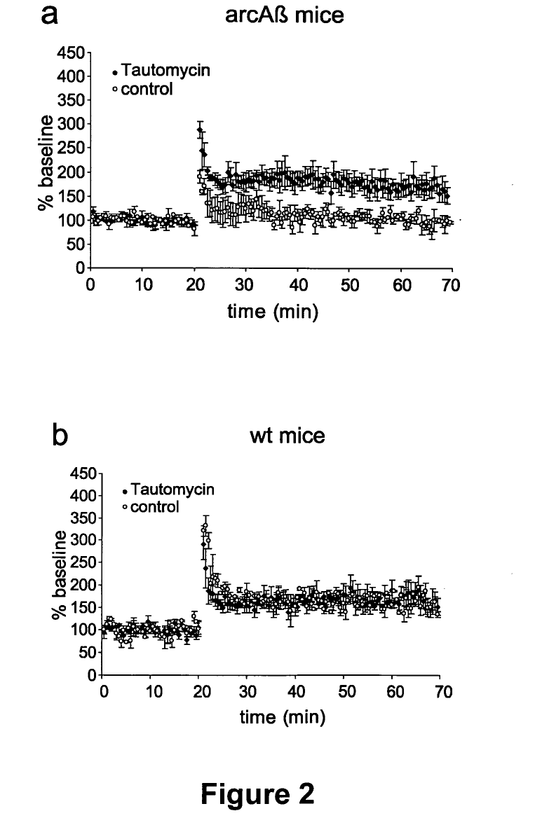

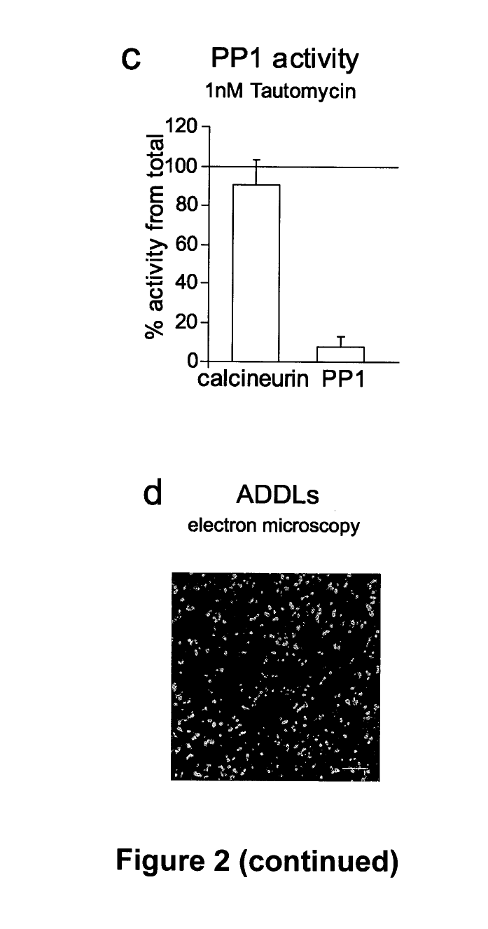

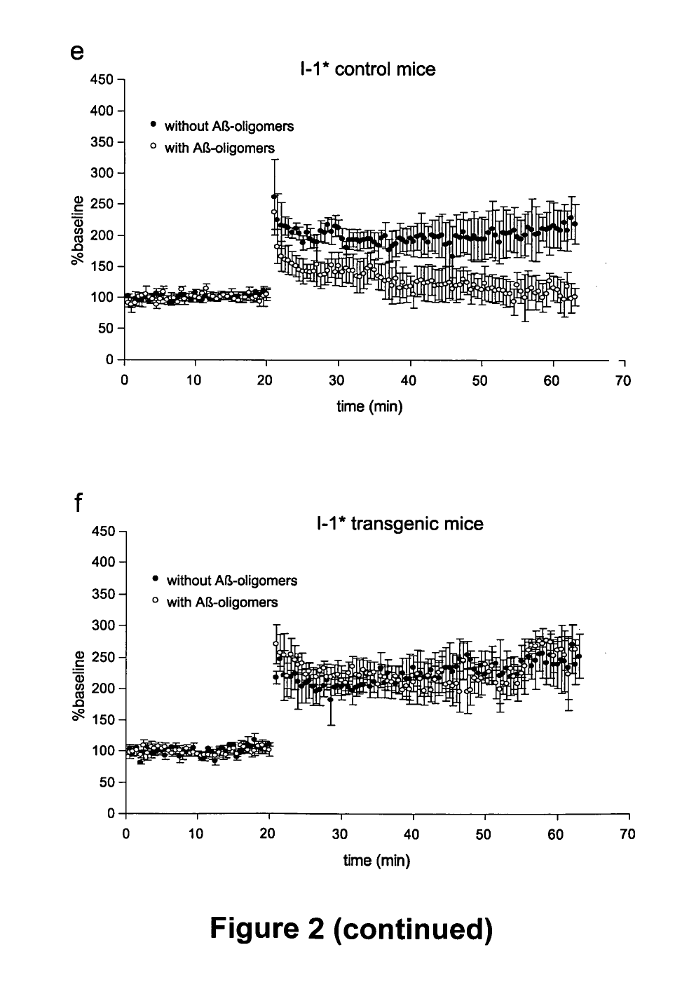

The fact that the LTP deficit is reversed by passive antibody transfer and that the expression of major signaling proteins is not altered in arcAβ mice suggests the possibility that a transient rather than persistent mechanism is involved. Because protein phosphorylation/dephosphorylation is itself reversible and transient and modulates neuronal signaling critical for synaptic plasticity (Sweatt, Curr. Biol. 11 (2001), R391-394), and because decreased protein phosphatase activity can reversibly affect LTP (Jouvenceau et al., Eur. J. Neurosci. 24 (2006), 564-572), it was tested whether altered protein phosphatase activity may be involved in the LTP deficit in arcAβ mice. Protein phosphatase 1 (PP1) was inhibited, one of the most abundant brain phosphatases known to negatively regulate synaptic plasticity (Genoux et al., Nature 418 (2002), 970-975; Jouvenceau et al., Eur. J. Neurosci. 24 (2006), 564-572) in acute slices from arcAβ mice with 1 nM tautomycin for at least 1 h before LTP measurements (supplementary methods). The inhibition of PP1 by tautomycin fully rescued the LTP deficit in arcAβ mice (FIG. 2a, p<0.05, repeated measurement ANOVA). Tautomycin had no effect on LTP in slices from non-transgenic mice (FIG. 2b, p=0.3), suggesting a rescue mechanism rather than a general enhancement of LTP. The effect of tautomycin on LTP was directly due to the inhibition of PP1 and did not involve calcineurin, another protein phosphatase that contributes to PP1 regulation, since activity assays revealed that 1 nM tautomycin inhibits about 90% of PP1 activity but less than 10% of calcineurin activity (FIG. 2c).

### Example 3

### Endogenous PP1 Inhibition Confers Resistance to Aβ Mediated Toxicity

The full rescue of LTP by the PP1 inhibitor tautomycin in arcAβ mice suggests that PP1 is recruited in the course of Aβ-mediated toxic functions. To investigate whether inhibition of endogenous PP1 prior to Aβ-treatment may confer a resistance to Aβ-mediated toxicity, advantage of a transgenic mouse model was taken, in which PP1 is selectively inhibited in forebrain neurons by expression of a constitutively active form of the endogenous PP1 inhibitor inhibitor −1 (I-1*) (Genoux et al., Nature 418 (2002), 970-975; Mansuy et al., Neuron 21 (1998), 257-265). Acute slices from adult I-1* transgenic mice were exposed to Aβ-oligomers produced from synthetic Aβ1-42 (supplementary methods). Electron microscopy images of the Aβ-oligomers preparation confirmed that it contained globular Aβ-assemblies of 7 to 12 nm diameter (FIG. 2d), consistent with previously described Aβ-oligomers (Klein, Neurochem. Int. 41 (2002), 345-352). To verify that the Aβ-oligomers preparation had the reported toxic effect on LTP (Wang et al., Brain Res. 924 (2002), 133-140), slices from wildtype mice were bathed in normal artificial cerebrospinal fluid (aCSF) or aCSF containing an Aβ-oligomers solution corresponding to 0.5 μM initial peptide (supplementary methods). As expected, LTP was inhibited by Aβ-oligomers returning to baseline level within 10 min after LTP induction (FIG. 3b). Likewise, slices from I-1* littermate controls showed Aβ-oligomers mediated LTP deficit (FIG. 2e, p<0.05, repeated measurement ANOVA). In contrast, however, Aβ-oligomers failed to inhibit LTP in slices from I-1* transgenic mice with reduced PP1 activity. In these mice, stable LTP was induced despite the presence of Aβ-oligomers (FIG. 2f, p=0.6, repeated measurement ANOVA comparing LTP with and without Aβ-oligomers).

Taken together, the data obtained in accordance with the present invention show that synaptic disturbance caused by Aβ-oligomers in vitro and in vivo is reversible, that it can be rescued by neutralizing antibodies or inhibition of PP1. The rescue of LTP and the immunity against Aβ-oligomer-mediated toxicity conferred by PP1 inhibition in arcAβ mice strongly suggest that PP1 is a key player in mediating Aβ-related toxicity. Therefore, PP1 is an interesting target for the development of novel therapeutic approaches designed to block Aβ-mediated toxicity in AD.

1. A pharmaceutical composition for the treatment, amelioration, or prevention of a disorder associated with amyloid β (Aβ) pathology/amyloidosis, said composition comprising an inhibitor of protein phosphatase 1 (PP1), and optionally a pharmaceutically acceptable carrier.

2. The pharmaceutical composition of claim 1, wherein said disorder is impairment of hippocampal long-term potentiation (LTP).

3. The pharmaceutical composition of claim 1, wherein the inhibitor is designed to be applied exogenously.

4. The pharmaceutical composition of claim 3, wherein said inhibitor is tautomycin or a derivative thereof.

5. The pharmaceutical composition of claim 1, wherein the inhibitor is designed to be expressed in a target cell or tissue.

6. The pharmaceutical composition of claim 5, wherein said agent is PP1 inhibitor (I-1*).

7. A method of diagnosis of a disorder associated with amyloid β (Aβ) pathology/amyloidosis, said method comprising:
(a) assaying a sample from a subject for PP1 gene product or activity; and
(b) determining the level of PP1 gene product or activity, wherein an altered level compared to a control indicates the presence of the disorder.

8. The method of claim 7, wherein the PP1 gene product is determined by a nucleic acid.

9. The method of claim 8, wherein the nucleic acid is labelled or otherwise modified.

10. The method of claim 7, wherein the PP1 gene product is determined by an antibody.

11. The method of claim 10, wherein the antibody is detectably labelled or otherwise modified.

12. The method of claim 10, wherein the PP1 gene product is detected by a secondary antibody.

13. A kit for use in a method of claim 7, said kit comprising an antibody or a nucleic acid probe and/or reagents suitable for the detection of PP1 activity.

14. A method of screening for or profiling of an inhibitor of PP1, said method comprising use of a non-human transgenic animal that expresses human APP carrying both familial Swedish and Arctic mutations.

15. The method of claim 14, wherein said animal is a mouse.

16. The pharmaceutical composition of claim 2, wherein the inhibitor is designed to be applied exogenously.

17. The pharmaceutical composition of claim 2, wherein the inhibitor is designed to be expressed in a target cell or tissue.

18. The method of claim 7, wherein said disorder is impairment of hippocampal long-term potentiation (LTP).

19. The method of claim 11, wherein the PP1 gene product is detected by a secondary antibody.
